(19) United States
(12) Patent Application Publication
(10) Pub. No.: US 2024/0113462 A1
(43) Pub. Date: Apr. 4, 2024

(54) SURGICAL INSTRUMENT WITH ELECTRICAL CONNECTION

(71) Applicant: Covidien LP, Mansfield, MA (US)

(72) Inventors: Andriy Buyda, Northford, CT (US); David M. Chowaniec, Rocky Hill, CT (US); Xingrui Chen, Glastonbury, CT (US); Brian J. Banerjee, Southington, CT (US); Saumya Banerjee, Southington, CT (US)

(21) Appl. No.: 18/539,557

(22) Filed: Dec. 14, 2023

Related U.S. Application Data
(63) Continuation of application No. 17/402,772, filed on Aug. 16, 2021, now Pat. No. 11,862,884.

(51) Int. Cl. H01R 13/24; A61B 17/072

(52) U.S. Cl. CPC H01R 13/2442; A61B 17/072; A61B 17/07207; A61B 2017/00477

(57) ABSTRACT

A surgical instrument includes a housing, an elongated portion, and a loading unit. The elongated portion extends distally from the housing, defines a longitudinal axis, and includes an electrical contact. The loading unit is configured to releasably engage the elongated portion. The loading unit includes an electronic component, a proximal portion including an electrical contact in electrical communication with the electronic component, and an end effector coupled to the proximal portion and configured to manipulate tissue. Engagement between the elongated portion and the loading unit causes the electrical contact of the elongated portion to engage the electrical contact of the loading unit thereby electrically connecting the elongated portion and the loading unit.

### CROSS-REFERENCE TO RELATED APPLICATIONS

This application is a continuation of U.S. patent application Ser. No. 17/402,772, filed Aug. 16, 2021, the entire disclosure of which is incorporated by reference herein.

### BACKGROUND

### Technical Field

The present disclosure relates generally to instruments for surgically manipulating tissue and, more specifically, to surgical instruments that form an electrical connection with a loading unit.

### Background of Related Art

Various types of surgical instruments used to surgically manipulate and join tissue are known in the art, and are commonly used, for example, for closure of tissue or organs in transection, resection, anastomoses, for occlusion of organs in thoracic and abdominal procedures, and for electrosurgically fusing or sealing tissue.

Surgical instruments may be operated manually, robotically, with powered components, or any combination thereof.

Additionally, a loading unit (e.g., a single use loading unit or a disposable loading unit) may be attached to an elongated or endoscopic portion of several types of surgical instruments. Such loading units allow surgical instruments to have greater versatility, for example. The loading units may be configured for a single use, and/or may be configured for multiple uses.

Further, some loading units include an identification chip and/or other electronic components. When such a loading unit is used with the remainder of the surgical instrument (e.g., when the loading unit having an identification chip is engaged with an elongated portion of a surgical instrument), it may be important to relay the information included on the identification chip of the loading unit to the surgical instrument engaged therewith. Thus, it may be suitable to provide a surgical instrument that can provide an electrical connection with a loading unit engaged therewith.

### SUMMARY

The disclosure relates to a surgical instrument comprising a housing, an elongated portion, and a loading unit. The elongated portion extends from the housing, defines a longitudinal axis, and includes an electrical contact. The loading unit is configured to releasably engage the elongated portion. The loading unit includes an electronic component, a proximal portion including an electrical contact in electrical communication with the electronic component, and an end effector coupled to the proximal portion and configured to manipulate tissue. Engagement between the elongated portion and the loading unit causes the electrical contact of the elongated portion to engage the electrical contact of the loading unit thereby electrically connecting the elongated portion and the loading unit.

In aspects, the electrical contact of the elongated portion may be movable toward and away from the longitudinal axis.

In aspects, the electrical contact of the elongated portion may include at least five linear segments. In disclosed aspects, three linear segments of the five linear segments of the electrical contact of the elongated portion may form a flat-bottom V-shape. In further disclosed aspects, the electrical contact of the elongated portion may be made from sheet metal.

In aspects, the electrical contact of the loading unit may be movable toward and away from the longitudinal axis.

In aspects, a distal end of the electrical contact of the loading unit may be fixed from movement relative to the longitudinal axis, and a proximal end of the electrical contact of the loading unit may be movable toward and any from the longitudinal axis.

In aspects, a proximal end of the electrical contact of the loading unit may form an acute angle with the longitudinal axis.

The disclose also relates to a surgical instrument configured to releasably engage a loading unit having an electronic component. The surgical instrument comprises a powered handle assembly, an elongated portion, and an electrical contact. The elongated portion extends distally from the powered handle assembly, defines a longitudinal axis, and includes an outer wall. The electrical contact is disposed within the outer wall of the elongated portion. At least a portion of the electrical contact is movable toward and away from the longitudinal axis. The electrical contact is configured to communicate with the electronic component of the loading unit when the surgical instrument is engaged with the loading unit.

In aspects, the electrical contact may include at least five linear segments. In disclosed aspects, three linear segments of the five linear segments of the electrical contact may form a flat-bottom V-shape. In further disclosed aspects, the electrical contact may be made from sheet metal.

In aspects, the surgical instrument may include at least one of a processor or a storage unit disposed in electrical communication with the electrical contact.

### BRIEF DESCRIPTION OF THE DRAWINGS

Various embodiments of the disclosure are illustrated herein with reference to the accompanying drawings, wherein:

FIG. 1 is a perspective view of a surgical instrument in accordance with an embodiment of the disclosure;

FIG. 2 is a perspective, assembly view of the surgical instrument of FIG. 1 illustrating a loading unit and part of an elongated portion;

FIG. 3 is an enlarged view of the area of detail indicated in FIG. 2, showing a proximal portion of the loading unit;

FIG. 4 is an enlarged view illustrating distal portions of the elongated portion engaged with proximal portions of the loading unit of the surgical instrument within the area of detail indicated in FIG. 1;

FIG. 5 is a perspective view illustrating portions of the loading unit and the elongated portion in a partially-engaged position; and

FIG. 6 is a perspective view illustrating portions of the loading unit and the elongated portion in a fully-engaged position.

### DETAILED DESCRIPTION

Embodiments of the disclosed surgical instrument are described in detail with reference to the drawings, wherein like reference numerals designate corresponding elements in each of the several views. As is common in the art, the term “proximal” refers to that part or component closer to the user or operator, e.g., surgeon or physician, while the term “distal” refers to that part or component farther away from the user.

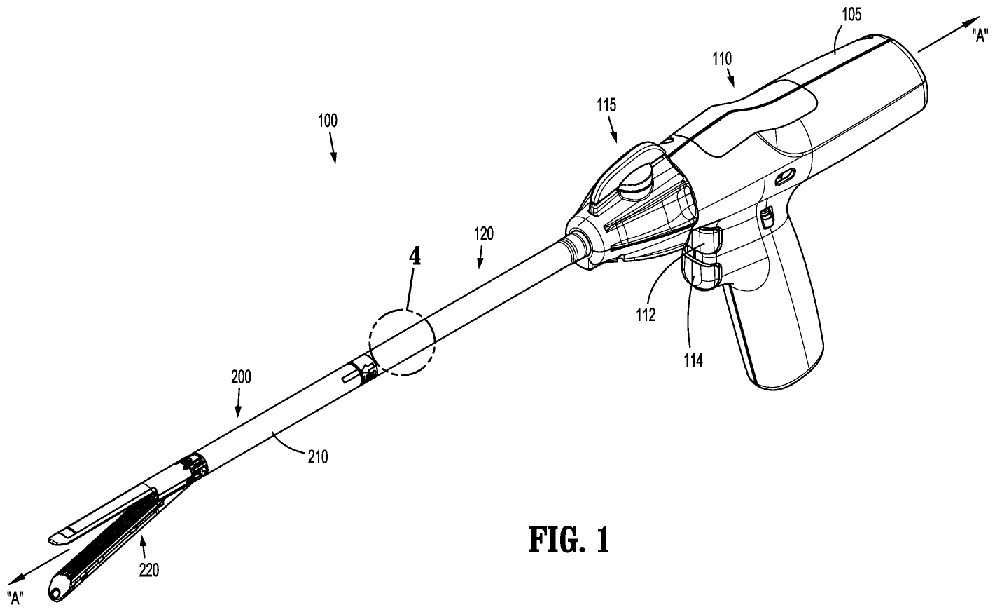

FIGS. 1-6 illustrate a surgical instrument in accordance with an aspect of the disclosure generally designated as reference numeral 100. While the surgical instrument 100 in the accompanying figures is depicted as a surgical stapling instrument, the surgical instrument 100 of the disclosure is not limited to a surgical stapling instrument; the surgical instrument 100 may be any suitable surgical instrument including but not limited to a vessel sealing instrument, a surgical grasper, a surgical clip applier, a circular stapling instrument, etc.

With particular reference to FIG. 1, the surgical instrument 100 includes a housing 105, a handle assembly 110, an adapter assembly 115, an elongated portion 120 extending distally from the adapter assembly 115 and defining a longitudinal axis “A-A,” and a loading unit 200 engaged with a distal end of the elongated portion 120. The loading unit 200 includes a proximal portion 210 and an end effector 220, and is releasably engageable with the elongated portion 120. While FIG. 1 illustrates the surgical instrument 100 including a powered handle assembly including a first actuator 112 and a second actuator 114, other types of handles can be used such as, for example, those including a pivotable handle, motor-driven, hydraulic, ratcheting, etc. As used herein, “handle assembly” encompasses all types of handle assemblies. Additionally, the surgical instrument 100 and components thereof are usable as part of a robotic surgical system.

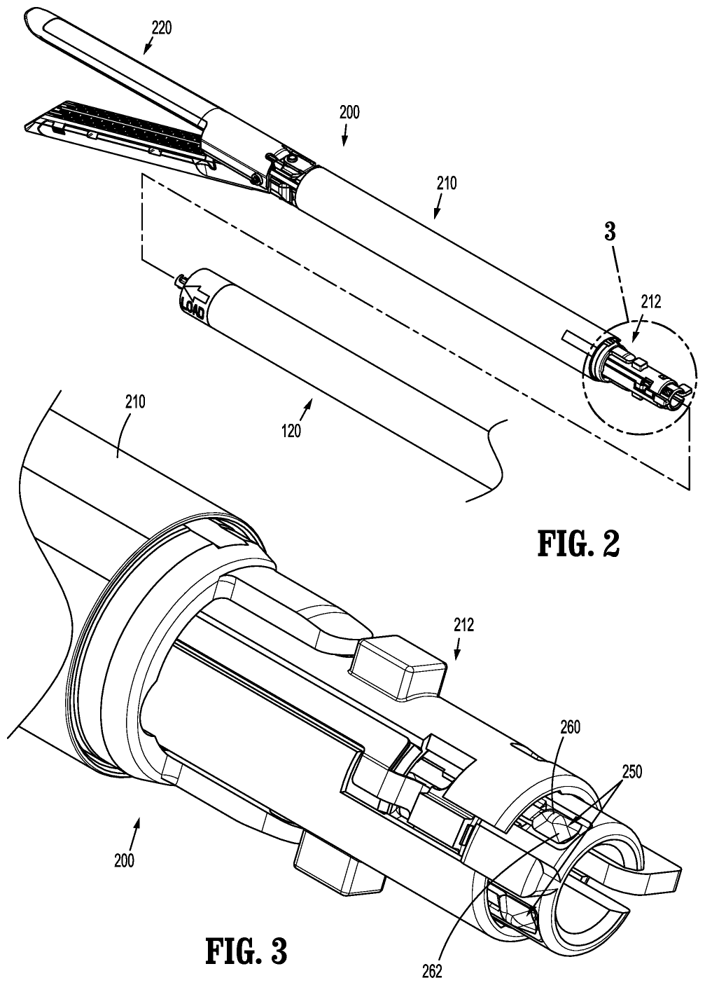

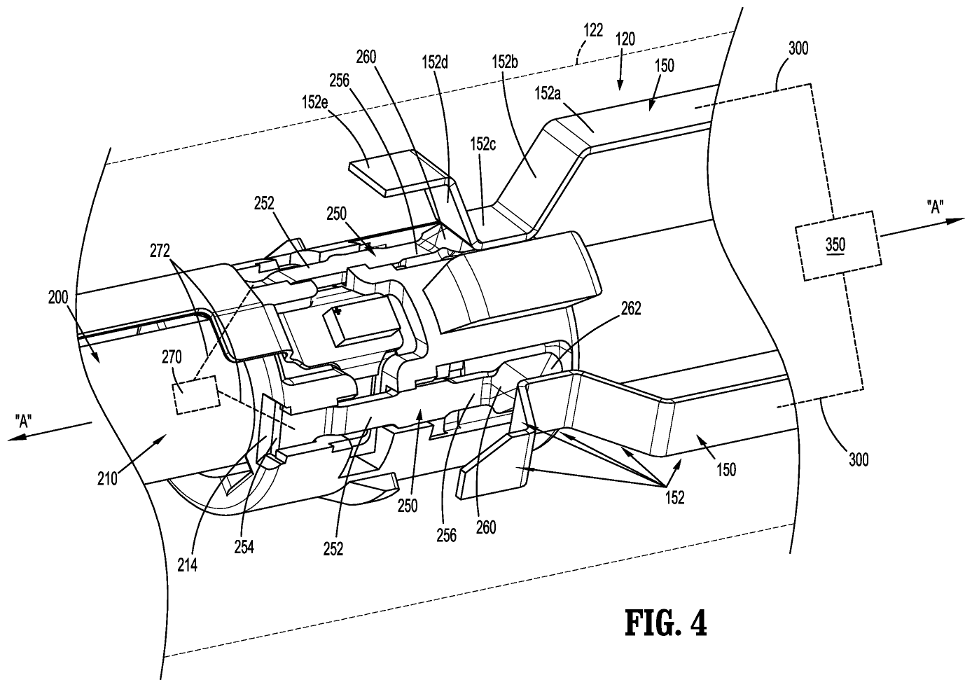

Referring now to FIGS. 2 and 3, details of the loading unit 200 are shown. The proximal portion 210 of the loading unit 200 includes a plurality of electrical contacts 250 adjacent a proximal end 212 thereof. Each electrical contact of the plurality of electrical contacts 250 is configured to store and relay information, and may either include or be in electrical communication with an electronic component 270 via leads 272, for instance (shown schematically in FIG. 4). The electronic component 270 may be a storage device, such as an EPROM or any suitable flash storage device, configured to store information relating to the type of end effector 220 (e.g., used for surgical stapling, vessel sealing, etc.) included on the loading unit 200, the length of the loading unit 200, the diameter of the loading unit 200, the number of fasteners included within the end effector 220, etc. In aspects, the electronic component 270 may be a sensor or an actuator.

With reference to FIG. 4, each electrical contact of the plurality of electrical contacts 250 includes an arm 252 and a finger 260. A distal end 254 of the arm 252 is engaged with a mounting portion 214 of the proximal portion 210 of the loading unit 200. The finger 260 of each electrical contact of the plurality of electrical contacts 250 extends proximally from a proximal end 256 of the arm 252. In this arrangement, the finger 260 and parts of the arm 252 of each electrical contact of the plurality of electrical contacts 250 are cantilevered thereby enabling portions of the plurality of electrical contacts 250 to flex toward and away from the longitudinal axis “A-A.” In aspects, the structure of the plurality of electrical contacts 250 biases the plurality of electrical contacts 250 radially outward away from the longitudinal axis “A-A.”

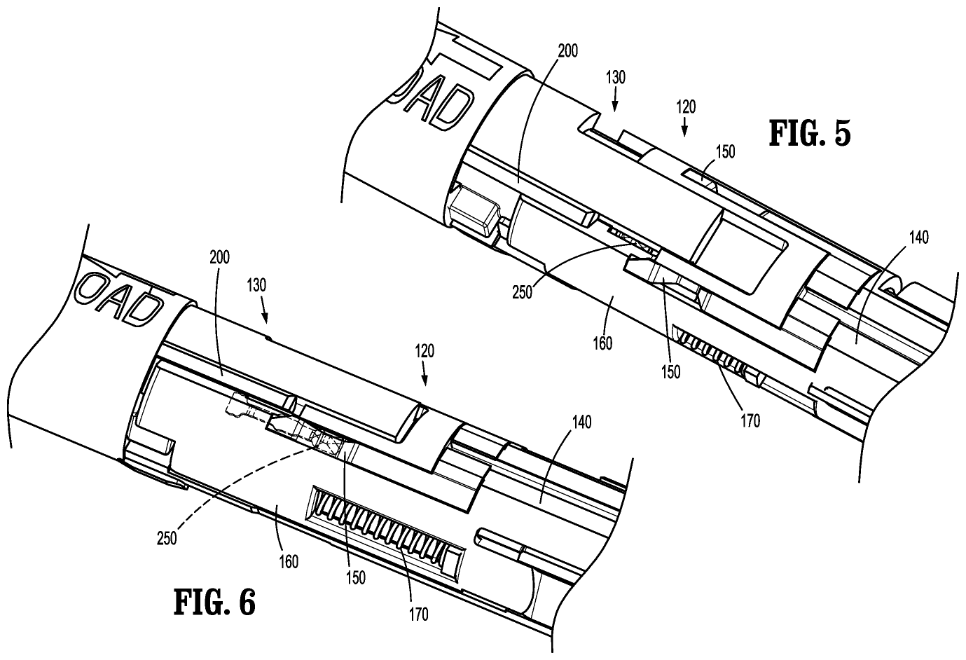

With particular reference to FIGS. 4-6, engagement between the loading unit 200 and the elongated portion 120 of the surgical instrument 100 is shown. For clarity, an outer wall 122 of the elongated portion 120 is shown in phantom in FIG. 4 and is omitted in FIGS. 5 and 6. The elongated portion 120 includes an engagement interface 130 that is configured to selectively engage portions of the loading unit 200. More particularly, the engagement interface 130 of the elongated portion 120 includes a base 140, a plurality of electrical contacts 150, a loading linkage 160, and a biasing element 170. In aspects, the number of electrical contacts of the plurality of electrical contacts 150 of the elongated portion 120 is equal to the number of electrical contacts of the plurality of electrical contacts 250 of the loading unit 200. In other aspects, the number of electrical contacts of the plurality of electrical contacts 150 of the elongated portion 120 is greater than or less than the number of electrical contacts of the plurality of electrical contacts 250 of the loading unit 200.

Referring to FIG. 4, each electrical contact of the plurality of electrical contacts 150 of the elongated portion 120 includes a plurality of segments 152. More particularly, in the illustrated aspect, each electrical contact of the plurality of electrical contacts 150 includes five segments 152: a first segment 152a, a second segment 152b, a third segment 152c, a fourth segment 152d, and a fifth segment 152e. Each electrical contact of the plurality of electrical contacts 150 may include more or fewer than five segments 152. Additionally, in the illustrated aspect, the first segment 152a, the third segment 152c, and the fifth segment 152e are parallel or generally parallel to each other and to the longitudinal axis “A-A,” and the second segment 152b and the fourth segment 152d are disposed at angles relative to their adjoining segments such that the second segment 152b, the third segment 152c, and the fourth segment 152d form a flat-bottom V-shape. Further, in aspects, the plurality of electrical contacts 150 is made of sheet metal.

Upon engagement between the loading unit 200 and the elongated portion 120 of the surgical instrument 100, the plurality of electrical contacts 250 of the loading unit 200 are moved proximally relative to the plurality of electrical contacts 150 of the elongated portion 120. Upon initial engagement, and as shown in FIG. 5, the plurality of electrical contacts 250 of the loading unit 200 and the plurality of electrical contacts 150 of the elongated portion 120 are free from physical contact with each other.

Upon continued and full engagement, and as shown in FIGS. 4 and 6, the plurality of electrical contacts 250 of the loading unit 200 and the plurality of electrical contacts 150 of the elongated portion 120 are in physical contact with each other. More particularly, in this position, the third segment 152c of each electrical contact of the plurality of electrical contacts 150 of the elongated portion 120 is in physical contact with the finger 260 of one electrical contact of the plurality of electrical contacts 250 of the loading unit 200 (see FIG. 4). This engagement is facilitated by the angled fourth segment 152d of the plurality of electrical contacts 150 of the elongated portion 120, by a ramped proximal portion 262 of the finger 260 of the plurality of electrical contacts 250 of the loading unit 200, and by the ability of both the plurality of electrical contacts 150 of the elongated portion 120 and the plurality of electrical contacts 250 of the loading unit 200 to be able to flex relative to the longitudinal axis “A-A,” for instance.

With reference to FIGS. 5 and 6, the base 140, the loading linkage 160, and the biasing element 170 are shown. FIG. 5 illustrates initial engagement between the loading unit 200 and the elongated portion 120. Here, the loading linkage 160 is in a proximal position relative to the base 140. In aspects, a portion of the base 140 may physically contact a portion of the loading linkage 160 to resist the distally-directed force of the biasing element 170. Additionally, as discussed above, in this initial engagement, the plurality of electrical contacts 150 of the elongated portion 120 are not in physical contact with the plurality of electrical contacts 250 of the loading unit 200.

FIG. 6 illustrates complete engagement between the loading unit 200 and the elongated portion 120. Here, the biasing element 170 has urged the loading linkage 160 to its distal position relative to the base 140. Moreover, as discussed above, in this complete or full engagement, each electrical contact of the plurality of electrical contacts 150 of the elongated portion 120 is in physical contact with one electrical contact of the plurality of electrical contacts 250 of the loading unit 200.

Additionally, upon proper engagement, the information stored on the plurality of electrical contacts 250 and/or the electronic component 270 of the loading unit 200 is electrically communicated through the plurality of electrical contacts 150 of the elongated portion 120, and through leads 300 (schematically illustrated in FIG. 4), to a processor and/or storage unit 350 (schematically illustrated in FIG. 4) which is engaged with the elongated portion 120, the housing 105, the handle assembly 110, and/or the adapter assembly 115, thereby allowing the surgical instrument 100 to receive the information stored on the loading unit 200.

While the above description contains many specifics, these specifics should not be construed as limitations on the scope of the present disclosure, but merely as illustrations of various embodiments thereof. Therefore, the above description should not be construed as limiting, but merely as exemplifications of various aspects. Those skilled in the art will envision other modifications within the scope and spirit of the claims appended hereto.

1. (canceled)

2. A surgical instrument configured to releasably engage a loading unit having an electronic component, the surgical instrument comprising:
a powered assembly;
an elongated portion coupled to the powered assembly, defining a longitudinal axis, and including an outer wall; and
an electrical contact disposed within the outer wall of the elongated portion, at least a portion of the electrical contact being movable toward and away from the longitudinal axis, the electrical contact having at least three segments including a first segment, a second segment, and a third segment, the second segment disposed distally of and being closer to the longitudinal axis than the first segment, and the third segment disposed distally of the second segment and being disposed at an angle relative to the longitudinal axis,
wherein the electrical contact is configured to communicate with the electronic component of the loading unit when the surgical instrument is engaged with the loading unit.

3. The surgical instrument according to claim 2, wherein at least a portion of the first segment of the electrical contact is parallel to the longitudinal axis.

4. The surgical instrument according to claim 3, wherein at least a portion of the second segment is parallel to the longitudinal axis.

5. The surgical instrument according to claim 2, wherein the electrical contact is made from sheet metal.

6. The surgical instrument according to claim 2, further including at least one of a processor or a storage unit disposed in electrical communication with the electrical contact.

7. The surgical instrument according to claim 2, wherein the third segment of the electrical contact is disposed at an angle with respect to the first segment.

8. The surgical instrument according to claim 7, wherein at least a portion of the first segment is parallel to at least a portion of the second segment.

9. The surgical instrument according to claim 7, wherein at least a portion of the first segment, and at least a portion of the second segment are parallel to the longitudinal axis.

10. The surgical instrument according to claim 2, wherein the third segment of the electrical contact is linear.

11. A surgical instrument configured to releasably engage a loading unit having an electronic component, the surgical instrument comprising:
an elongated portion defining a longitudinal axis, and including an outer wall; and
an electrical contact disposed within the outer wall of the elongated portion, at least a portion of the electrical contact being movable toward and away from the longitudinal axis, the electrical contact having at least three segments including a first segment, a second segment, and a third segment, the second segment disposed distally of and being closer to the longitudinal axis than the first segment, the second segment disposed at an angle relative to the longitudinal axis, and the third segment disposed distally of the second segment,
wherein the electrical contact is configured to communicate with the electronic component of the loading unit when the surgical instrument is engaged with the loading unit.

12. The surgical instrument according to claim 11, wherein at least a portion of the first segment of the electrical contact is parallel to the longitudinal axis.

13. The surgical instrument according to claim 12, wherein at least a portion of the third segment is parallel to the longitudinal axis.

14. The surgical instrument according to claim 11, wherein the electrical contact is made from sheet metal.

15. The surgical instrument according to claim 11, further including at least one of a processor or a storage unit disposed in electrical communication with the electrical contact.

16. The surgical instrument according to claim 11, wherein the third segment of the electrical contact is parallel to the first segment.

17. The surgical instrument according to claim 11, wherein the second segment of the electrical contact interconnects the first segment and the third segment.

18. The surgical instrument according to claim 11, wherein the third segment of the electrical contact is linear.

19. The surgical instrument according to claim 11, wherein the at least three segments of the electrical contact includes a fourth segment, the fourth segment disposed distally of the third segment.

20. The surgical instrument according to claim 19, wherein a distal portion of the fourth segment of the electrical contact is positioned farther away from the longitudinal axis than a proximal portion of the fourth segment.

21. The surgical instrument according to claim 20, wherein a distal portion of the second segment of the electrical contact is positioned closer to the longitudinal axis than a proximal portion of the second segment.
